(19) United States
(12) Patent Application Publication
(10) Pub. No.: US 2020/0332424 A1
(43) Pub. Date: Oct. 22, 2020

(54) ELECTROLYSIS-TYPE HYDROGEN GAS SUCTION TOOL

(71) Applicant: Aqua Bank CO.,LTD., Osaka-shi, Osaka (JP)

(72) Inventor: Takashi TAKEHARA, Osaka-shi, Osaka (JP)

(21) Appl. No.: 16/305,624

(22) PCT Filed: Sep. 7, 2017
(86) PCT No.: PCT/JP2017/032197

§ 371 Date: Nov. 29, 2018

(30) Foreign Application Priority Data
Sep. 11, 2016 (JP) 2016-177193

(51) Int. Cl. C25B 1/04; C25B 11/03; C25B 15/08; C25B 9/06; A61L 9/03

(52) U.S. Cl. CPC C25B 1/04; C25B 11/03; A61L 9/03; C25B 9/06; C25B 15/08

(57) ABSTRACT

Provided is an electrolysis-type hydrogen gas suction tool which is portable and easily supply a predetermined amount of hydrogen gas. The suction tool is provided with a main body cover member having a battery, a control board which controls an electric power supply from the battery, and a pair of positive and negative electrodes which are applied current in relation to a positive electrode and a negative electrode of the battery or are shielded the current application thereof on the basis of the control board, a water storable electrolysis tank which is detachably attached to the main body cover member and has a lower portion in which the pair of positive and negative electrodes is inserted inside under the attached state, a nozzle which has a through hole, and a mixing portion which has a flow channel fluidly connecting the nozzle portion and an upper end of the electrolysis tank.

### TECHNICAL FIELD

The present invention relates to an electrolysis-type hydrogen gas suction tool which is portable and can easily supply a predetermined amount of hydrogen gas.

### BACKGROUND ART

In recent years, there have been provided hydrogen effectiveness in various animal disease experiments such as a neurodegenerative and an acute lung injury, and human clinical tests in metabolic syndrome and diabetes, and various researches have been actively carried out in a medical application. Hydrogen removes only bad active oxygen (hydroxyl radical) which causes promotion of aging and various diseases such as arteriosclerosis and cancer, from an inside of the body, and does not adversely affect tissue and cell in the body. A wide range of methods are employed for taking hydrogen into the body, for example, intravenous dosage, oral administration of aqueous solution and gas inhalation.

In order to prevent the aging in various states such as during exercise, during eating and drinking, during smoking, during staying under ultraviolet light and contaminated environment, and during being highly stressed due to lack of sleep and long-hour working, in which the active oxygen particularly tends to be generated in the body, and promote beauty and health, it is suggested to take hydrogen in the body. In particular, taking into consideration a market expansion of a pseudo electronic cigarette and a cigarette which does not discharge any secondhand smoke in recent years, the potential needs of smoking hydrogen which derives the health promotion is deemed to be great.

Here, roughly two methods have been provided as the conventional hydrogen generation method. First of all, a method which utilizes a hydrogen generation chemical reaction such as a chemical reaction between magnesium particles or aluminum particles and water has been known as a simple method. In this method, a case for hydrogen generation which chemically reacts the magnesium particles and water is provided, for example, as in patent literature 1. In this instance of the case for hydrogen generation, there is employed a structure which performs the hydrogen generation reaction in an inner portion and discharges only the hydrogen to an outside through a hydrogen permeable film disposed in the case, thereby being compact and portable.

However, in the hydrogen generation case in the patent literature 1 mentioned above, the case is merely placed into a container such as a plastic bottle and is used for the purpose of changing moisture in an inner portion thereof into hydrogen water and setting the hydrogen water to drinking water, but the case cannot be utilized as a hydrogen gas suction tool such as smoking supplies as it is. Further, even if the structure of the hydrogen generation case in the patent literature 1 is design changed for the hydrogen suction tool into a structure which can directly suck, a step of opening the case for pouring water when using and causing reaction between the magnesium particles in the inner portion with water is essential. As a result, a troublesome work is required for the user. Specifically, it is necessary for the user to prepare water for pouring, and spend energy for a case opening work when pouring water and a water pouring work, and water wetting may occur when pouring water.

Further, a water electrolysis method can be thought as a second hydrogen generation method, and this is merely a method of generating the hydrogen water. For example, this method employs a hydrogen generation device such as a desktop type hydrogen generation device which can generate the hydrogen gas by putting water into an electrolysis tank in which an electrolysis plate having an ion exchange membrane, a pair of electrode plates closely adhered respectively to both surfaces of the ion exchange membrane, and a stationary portion making the pair of electrode plate be closely adhered respectively to both surfaces of the ion exchange membrane is mounted, and applying current to the electrolysis plate (refer, for example, to patent literature 2). In this hydrogen generation device, since the user can use while optionally moving, usability is improved in comparison with the hydrogen generation device which can be used only in a stationary state.

However, the hydrogen generation device such as the desktop type as mentioned above is downsized to some degree, but does not get to such a compact size that the user can put in a bag and carry, and it is necessary to prepare a power source from an electric outlet for using. As a result, this device has a restriction in a moving range for making good use of the device as a hydrogen gas suction tool with which the user sucks the hydrogen gas inside of the body. Further, in the instance of using a battery having a power source built-in as a technical problem, it is essential to prepare a space for embedding the battery and to shield from water between the electrolysis tank and the battery, so that there is a limit to downsize. In addition, a lot of user cannot freely get due to its expensiveness and the device is not suitable for outside use.

### CITATIONS LIST

### Patent Literature

Patent Literature 1: JP2004-41949 A

Patent Literature 2: JP2014-019640 A

### SUMMARY OF INVENTION

### Technical Problems

The present invention is made by taking the above circumstances into consideration, and an object of the present invention is to provide an electrolysis-type hydrogen gas suction tool structured such that a sufficient amount of hydrogen gas generation can be secured even in a state in which the moisture within the electrolysis tank is reduced, while securing a space for embedding a battery and securing a water shielding between an electrolysis tank and the battery in spite that the tool is inexpensive and has a rechargeable battery and is compact for enabling the user to bring and freely carry with.

### Solutions to Problems

In order to solve the problems mentioned above, an electrolysis-type hydrogen gas suction tool according to the present invention includes:

a main body cover member (for example, a main body cover 1 in an embodiment of the present description) having a battery, a control board which controls an electric power supply from the battery, and a pair of positive and negative electrodes (for example, a mesh electrode 17 in the embodiment of the present description) which are applied current in relation to a positive electrode and a negative electrode of the battery or are shielded the current application thereof on the basis of the control board;

a water storable electrolysis tank (for example, an electrolysis tank 10 and an electrolysis tank lid 12 in the embodiment of the present description) which is detachably attached to the main body cover member and has a lower portion in which the pair of positive and negative electrodes is inserted inside under the attached state;

a nozzle portion (for example, a nozzle 5 in the embodiment of the present description) which has a through hole; and

a mixing portion (for example, a mixer 2 in the embodiment of the present description) which has a flow channel fluidly connecting the nozzle portion and an upper end of the electrolysis tank,

wherein the electrolysis tank is constructed by an upper portion (for example, a water storage main body portion 46 in the embodiment of the present description) and a lower portion (for example, a diameter-reduced portion 45 in the embodiment of the present description) which are fluidly connected in their inner portions and are integrally formed, has a narrowing width from a side portion at one side to a side portion at the other side in the upper portion toward a boundary between the upper portion and the lower portion, and is provided with a partition plate (for example, a partition plate 45c in the embodiment of the present description) disposed from the vicinity of the boundary in the other side toward the one side.

According to the present electrolysis-type hydrogen gas suction tool, a diameter of the side portion in the electrolysis tank is reduced or narrowed from the one side, from the upper portion toward the lower portion, and the partition plate is provided in the inner portion in the other side. As a result, even in the case that water within the electrolysis tank is reduced due to the electrolysis and whichever width direction the tank is inclined, the narrowed boundary portion or the partition plate will function as a baffle plate so as to position an air layer in the upper portion and the lower portion is filled with water. Therefore, the pair of positive and negative electrodes can be immersed into water. Accordingly, it is possible to always secure the maximum hydrogen generation output.

Further, the pair of positive and negative electrodes of the electrolysis-type hydrogen gas suction tool according to the present invention is preferably arranged over all the areas in the vertical and width directions of the lower portion of the electrolysis tank.

According to the electrolysis-type hydrogen gas suction tool, since an air layer is not formed within the lower portion of the electrolysis tank in which the pair of positive and negative electrodes is positioned whichever width direction the tool is inclined, it is possible to always perform the hydrogen generation reaction at the maximum water storage amount of the lower portion by forming such a shape that the positive and negative electrodes is expanded up to all the area of the lower portion in which water is filled.

Further, it is preferable that the side portions at one side of the upper portion and the lower portion of the electrolysis tank are formed into an approximately step shape, the side portions at the other side of the upper portion and the lower portion of the electrolysis tank are continuously formed into a single side portion, and the lower portion of the electrolysis tank is fitted and inserted into an electrolysis tank reception portion in the vertical direction which is provided in a side portion of the main body cover member.

In this electrolysis-type hydrogen gas suction tool, the lower portion can be detachably and easily inserted into the main body cover member in the vertical direction by forming the lower portion narrower than the upper portion so as to form the side portion at the one side into the step shape, and the upper portion of the electrolysis tank can be arranged with the integral shape in the side portion of the main body cover.

Further, the electrolysis tank is specifically formed, in a cross sectional view in the vertical direction, into the following shape:

the upper portion thereof is formed into an approximately trapezoidal shape having legs which are inclined from an upper bottom to a lower bottom in the lower portion side of the electrolysis tank by setting both sides of the electrolysis tank in the width direction respectively to the upper bottom and the lower bottom which is longer than the upper bottom;

the lower portion is formed into an approximately trapezoidal shape having legs which are inclined from an upper bottom to a lower bottom in the upper portion side of the electrolysis tank by setting both sides of the electrolysis tank in the width direction respectively to the lower bottom and the upper bottom which is longer than the lower bottom; and the legs inclined from the upper bottom to the lower bottom in the upper portion and the lower portion of the electrolysis tank are approximately in common with the lower bottom of the upper portion and the upper bottom of the lower portion, and a configuration example is provided by setting a portion connecting the lower bottom in the lower portion and the upper bottom in the upper portion of the electrolysis tank in the leg to a bottom surface in the upper portion of the electrolysis tank for closing from an outside, setting a portion connecting the lower bottom in the upper portion and the upper bottom in the lower portion of the electrolysis tank in the leg to a bottom surface in the upper portion of the electrolysis tank for closing from the outside, and providing a partition plate extending to an intermediate position of the lower bottom in the lower portion of the electrolysis tank along the leg from the lower bottom in the lower portion of the electrolysis tank.

Further, in the above configuration example, it is preferable to arrange a battery reception portion, the electrolysis tank reception portion and the control board of the main body cover member side by side in the vertical direction.

According to this example, it is possible to downsize by arranging the electrolysis tank reception portion receiving the lower portion of the electrolysis tank, the battery reception portion receiving the battery and the control board in the main body cover member side by side in the vertical direction, and a portability is improved.

Further, the above configuration example may be provided with an aromatic heater member (for example, an aromatic heater portion 32 in the embodiment of the present description) which generates air with aroma by an electric power supply above the battery reception portion of the main body cover member.

It is possible to respond to potential needs of a user who desires to suck the hydrogen gas with aroma, and the preference is improved. Further, it is possible to provide a product which the user using the existing electronic cigarette with aroma can replace as a product having a further health promotion function without any sense of discomfort.

Further, in the case that the aromatic heater portion is mounted,

when the control board receives an operation signal requesting the hydrogen gas generation from the user and an operation signal requesting the air with aroma generation from the user, the control board controls the electric power supply and shutting off to the pair of positive and negative electrodes and to the aromatic heater member on the basis of the respective signals, and

the control board preferably controls so as to supply the electric power from the battery to the aromatic heater member under a condition that the electric power from the battery is supplied to the pair of positive and negative electrodes.

There are various users, and even the same user may have different preferences such that the user prefers to suck odorless hydrogen gas in some occasions but prefers to such hydrogen gas with aroma in other occasions. The tool can respond to the various preferences of the user mentioned above. Further, since the hydrogen gas cannot be visually recognized, the control condition is more preferably set in such a manner that the aromatic air is generated on the condition of the generating state of the hydrogen gas for preventing only the aromatic air from being sucked.

Further, a mixing portion may be provided, the mixing portion being structured such as to be installed to the bottom portion of the nozzle as well as being installed to the above of the electrolysis tank, and have a hydrogen gas flow channel which fluidly connects from the inner portion of the electrolysis tank to the through hole of the nozzle.

In the case of this electrolysis-type hydrogen gas suction tool, it is possible to make good use of a member (the mixing portion) serving as a lid member of the electrolysis tank as a guide member of the hydrogen gas to the nozzle. Therefore, it is possible to achieve the downsizing without waste.

Further, the mixing portion is preferably installed above the electrolysis tank and above the aromatic heater and provided with an aromatic air flow channel which fluidly connects from the aromatic heater member to the through hole of the nozzle, and

the hydrogen gas flow channel and the aromatic air flow channel are preferably structured such as to be joined together and be guided to the through hole of the nozzle.

The mixing portion is structured such that the flow channel from the aromatic heater portion and the hydrogen gas flow channel are joined together. Therefore, it is possible to further downsize, and have high decorativeness.

Further, the electrolysis-type hydrogen gas suction tool is preferably provided with a regulation valve (for example, an umbrella valve 23 in the embodiment of the present description) which is opened when the negative pressure is applied to the mixing portion side from the electrolysis tank to the hydrogen gas flow channel of the mixing portion, and is closed when the negative pressure is not applied.

The user can suck the hydrogen gas only when the user desires, and the hydrogen gas can be accumulated in the electrolysis tank when the user does not suck. As a result, it is possible to provide the hydrogen gas having a desired concentration when sucking.

### Advantageous Effects of Invention

According to the present invention, it is possible to provide an inexpensive hydrogen gas suction tool which the user can bring and freely carry with and can easily and sanitarily suck hydrogen when using. According to this hydrogen gas suction tool, the user can suck hydrogen gas into a mouth until the end of the reaction only by bending the hydrogen generation tool with the user's hand power when using, a water pouring work is not necessary and the liquid leakage to the user does not occur. Further, the hydrogen generation tool is convenient for carriage and inventory control and can be a simple and disposable product.

### BRIEF DESCRIPTION OF DRAWINGS

FIG. 1 is an assembly exploded view exemplifying each of members of an electrolysis-type hydrogen gas suction tool according to the present invention.

FIGS. 2(a), 2(b), 2(c), 2(d) and 2(e) show views of the electrolysis-type hydrogen gas suction tool in FIG. 1 as seen from respective directions, in which FIG. 2(a) shows a left side elevational view, FIG. 2(b) shows a front elevational view, FIG. 2(c) shows a right side elevational view, FIG. 2(d) shows a bottom elevational view, and FIG. 2(e) shows a top elevational view.

FIG. 3 shows a cross sectional view of the electrolysis-type hydrogen gas suction tool in FIGS. 1 to 2 along line A-A in FIG. 2(c).

FIGS. 4(a) and 4(b) shows the electrolysis-type hydrogen gas suction tool in FIGS. 1 to 3, in which FIG. 4(a) shows a perspective view as seen from the above in a left near side on the page space in FIG. 2(b), and FIG. 4(b) shows a perspective view as seen from the above in a right near side on the page space in FIG. 2(b).

### DESCRIPTION OF EMBODIMENTS

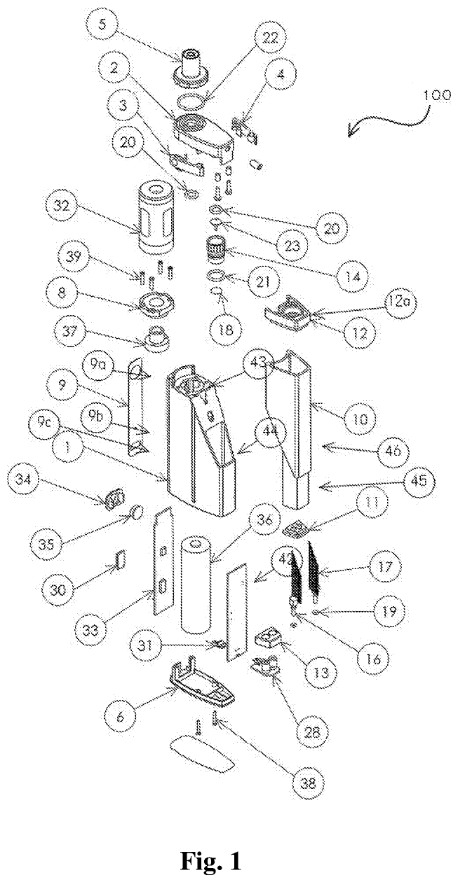

A description will be in detail given below of a representative embodiment according to an electrolysis-type hydrogen gas suction tool of the present invention with reference to FIGS. 1 to 4. However, it goes without saying that the present invention is not limited to the illustrated embodiment. Further, each of the drawings is provided for conceptually describing the present invention and may be shown in an exaggerated manner or a simplified manner in dimensions, ratios or numbers as occasion demand for easy understanding. Further, in the following description, the same reference numerals are attached to the same or corresponding portions, and an overlapping description may be omitted.

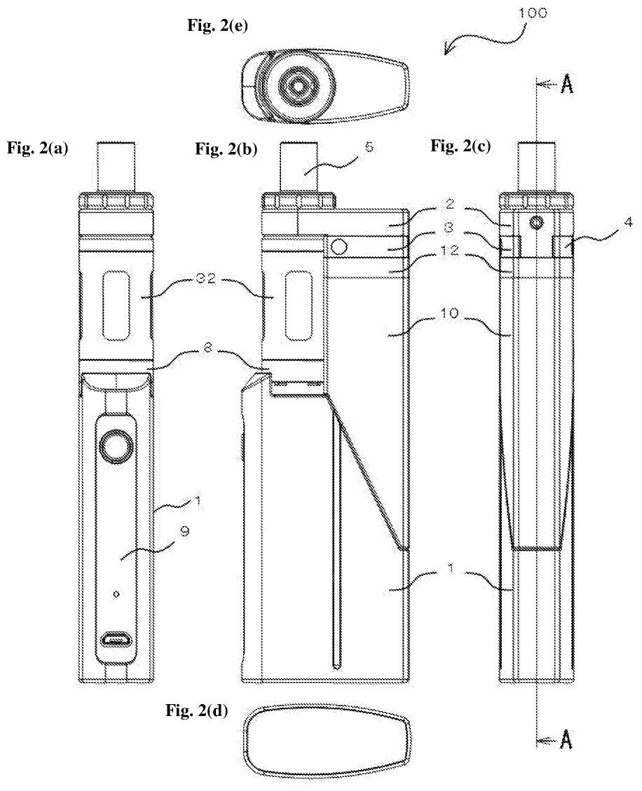

FIG. 1 is an assembly exploded view exemplifying each of members of an electrolysis-type hydrogen gas suction tool 100 according to the present invention. Further, FIGS. 2(a), 2(b), 2(c), 2(d) and 2(e) show views of the electrolysis-type hydrogen gas suction tool 100 in FIG. 1 as seen from respective directions, in which FIG. 2(a) shows a left side elevational view, FIG. 2(b) shows a front elevational view, FIG. 2(c) shows a right side elevational view, FIG. 2(d) shows a bottom elevational view, and FIG. 2(e) shows a top elevational view. In the present description, an up-and-down direction and a vertical direction mean an up-and-down direction on the page space and a vertical direction on the page space in FIG. 2(b), and a width direction, a lateral direction and a side portion side mean a right-and-left direction on the page space, a lateral direction on the page space and a right-and-left side portion side on the page space in FIG. 2(b).

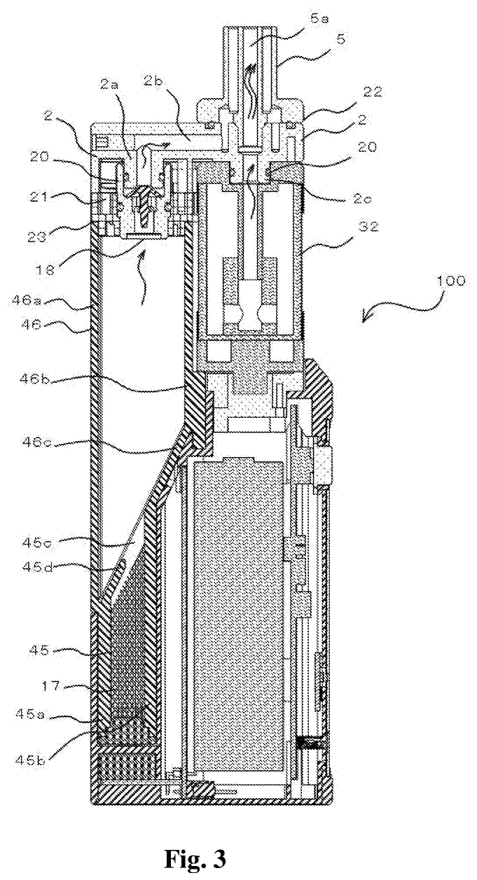

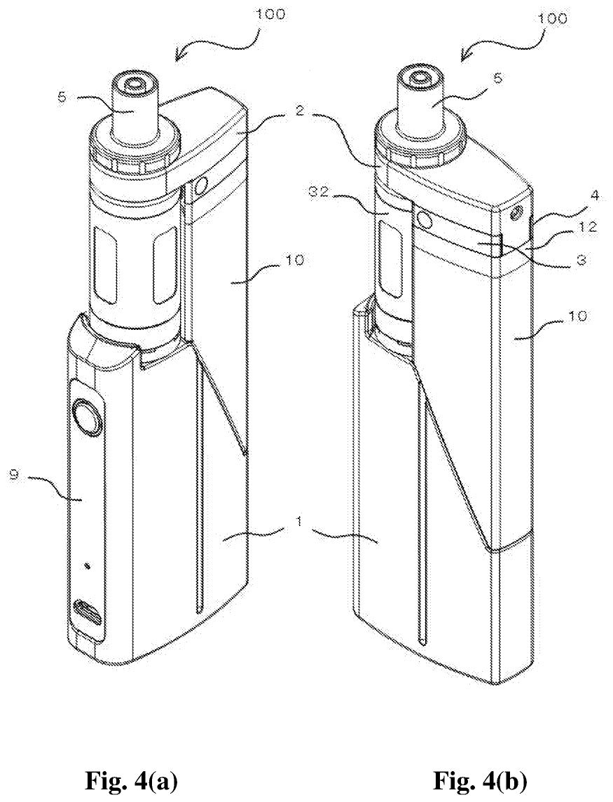

Further, FIG. 3 shows a cross sectional view of the electrolysis-type hydrogen gas suction tool 100 in FIGS. 1 to 2 along line A-A in FIG. 2(c). Further, FIGS. 4(a) and 4(b) shows the electrolysis-type hydrogen gas suction tool 100 in FIGS. 1 to 3, in which FIG. 4(a) shows a perspective view as seen from the above in a left near side on the page space in FIG. 2(b), and FIG. 4(b) shows a perspective view as seen from the above in a right near side on the page space in FIG. 2(b).

A description will be given below of the electrolysis-type hydrogen gas suction tool 100 mainly with reference to the assembly exploded view in FIG. 1 and further with reference to the other drawings for the purpose of illustration.

As mentioned above, FIG. 1 shows a configuration example of each of the members of the present hydrogen gas suction tool 100. A main body cover 1 is a resin case having a battery reception portion 43 which is open upward, through which a battery 36 is inserted and embedded in a vertical direction from the opening, and an electrolysis tank reception portion 44 which is disposed in parallel with the battery reception portion 43 in a vertical direction and has such a shape that a diameter-reduced portion 45 in a lower portion of the electrolysis tank 10 can be inserted and fitted from the above. The battery 36 used here is preferably a rechargeable lithium battery.

The main body cover 1 has a shape which is long in the side of the battery reception portion 43 and is cut in such a manner that an upper portion is inclined laterally in the side of the electrolysis tank reception portion 44. In a bottom portion of the main body cover 1, a bottom portion of the battery reception portion 43 can be opened and closed by using a main body bottom cover 6 as a lid member, and the bottom portion of the battery reception portion 43 is closed by the main body bottom cover 6 after the battery 36 is inserted from the bottom portion when assembling. The main body bottom cover 6 is closed by a cross recessed head screw 38. Further, the main body cover 1 is provided with a space in which two control boards (electronic substrates) 33 and 42 are arranged in such a manner as to sandwich the battery 36 in both sides of a side portion of the battery reception portion 43 in the vertical direction, and the control board 33 in a side surface side of the main body cover 1 is a main control board, and controls an electric power supply from the battery 36 to the control board 42 in the electrolysis tank 10 side which supplies electric power to a suction portion 32 (an aroma generating device) and a mesh electrode 17 (an electrode plate).

A decorative laminate 9 is installed to the side surface of the main body cover 1 along a side surface in a longitudinal direction, and the decorative laminate 9 is provided with a button hole 9a through which an operation button 35 for the control board 33 can be seen, an LED hole 9b for light irradiation from an LED substrate 30, and a charge connector hole 9c for connecting a connector charging the battery 36 from an external power source, in this order from the above.

When pushing the operation button 35 three times, an electric power supply signal is transmitted to the control board 42 in the control board 33, and the electric power of the battery 36 is supplied to a pair of mesh electrodes (electrode plates) 17 via a substrate connector housing 31 and a pressure-bonding substrate 28 for a predetermined period of time. When the electric power is supplied to the mesh electrode 17, an electric power supply signal is transmitted to the LED substrate 30 in the control board 33, and the LED substrate 30 makes the LED emit light. As a result, a user can visually recognize a hydrogen gas generating state via the LED hole 9b. Pushing the operation button 35 three times is set to a condition for supplying the power source to the mesh electrode 17 because this condition is set as a safety condition for avoiding an erroneous electric power supply by unintentionally operating the button when the user puts the hydrogen gas suction tool 100 in a pocket while moving.

The two paired mesh electrodes 17 are arranged side by side longitudinally toward the above, respectively form positive and negative electrodes, and correspond to the electric power from the positive and negative electrodes of the battery 36. Further, an upper end of the mesh electrode 17 has a shape which is diagonally cut out in such a manner as to correspond to a boundary line between the diameter-reduced portion 45 and the water storage main body portion 46 of the electrolysis tank 10. A lower end of the mesh electrode 17 is raised up on a terminal substrate 28 and a rod-shaped titanium electrode 16 is coupled thereto in such a manner as to achieve an electrical connection. In order to shield the mesh substrate 17 and the terminal substrate 28 from water in a state in which the mesh electrode 17 is raised, there are provided a packing 13 (made of a resin such as silicone) which is installed on the terminal substrate 28, and an O-ring (made of a resin such as silicone, hereinafter refer also to as O-ring) which is attached to a periphery of the titanium electrode 16.

The electrolysis tank 10 is a container for water storage, and a diameter-reduced portion 45 and a water storage main body portion 46 are integrally formed in this order from the below and are fluidly connected to each other internally. The water storage main body portion 46 can open upward and water can be poured therefrom, and is semi-closed by attaching the electrolysis tank lid 12 thereto. The electrolysis tank lid 12 passes through up and down, and is provided with a through opening 12a which receives the umbrella valve 23 and a screw cap 14. The water storage main body portion 46 includes an outside portion 46a which forms an approximately flat side wall in a lateral direction from an upper end to a lower end and is coupled to an upper end of the diameter-reduced portion 45 as it is as shown in FIG. 3; an inside portion 46b in the main body cover 1 side which is formed in parallel to the outside portion 46a from an upper end to a central lower position; and a bottom portion 46c which is bent and inclined from the central lower position. The bottom portion 46c extends to an intermediate position in a lateral direction and is coupled to the upper end of the diameter-reduced portion 45.

Further, the diameter-reduced portion 45 is thinner than the water storage main body portion 46 as mentioned above, the upper end of the outside portion 46a in the side wall side is continuously connected as it is to the lower end of the outside portion 46a of the water storage main body portion 46 and extends to the lower end as shown in FIG. 3, and the upper end of an inside portion 45b of the diameter-reduced portion 45 in the main body cover 1 side is bent downward at a position in a leading end (an edge portion) of the bottom portion 46c of the water storage main body portion 46 and is connected so as to extend to the lower end in parallel to the inside portion 45b.

Further, at a connection position between the lower end of the outside portion 46a in the water storage main body portion 46 and the upper end of the outside portion 46a in the diameter-reduced portion 45, there is provided a partition plate 45d which is inclined approximately in the same manner as the bottom portion 46c of the water storage main body portion 46 and extends to the opening 45c. The partition plate 45d extends over an entire inner portion in a vertical direction on the page space of FIG. 3. Therefore, even in the case that the aqueous solution accumulated in the electrolysis tank 10 is electrolyzed and the water storage amount is reduced, water is always accumulated approximately in the entire area of the inner portion of the diameter-reduced portion 45. Specifically, even when the water storage amount is reduced and the air layer is generated partly within the electrolysis tank 10, the diameter-reduced portion 45 is filled with water in the normal standing state and the air layer is not generated until water storage amount is reduced greatly since the diameter-reduced portion 45 is first of all thinner than the water storage main body portion 46.

The air layer may be generated within the diameter-reduced portion 45 in the case that the present hydrogen gas suction tool 100 is inclined or transversely placed. However, even in the case that the water storage amount is reduced to some degree, water is filled in the diameter-reduced portion 45 in the case of the present electrolysis tank 10. Specifically, in the case of being inclined in the leftward direction on the page space of FIG. 3, the bottom portion 46c serves as a baffle plate and the air layer is formed in the inside portion 46b side of the water storage main body portion 46. On the contrary, in the case of being inclined to the rightward direction on the page space of FIG. 3, the partition plate 45d serves as a baffle plate and the air layer is formed only in the outside portion 46a side of the water storage main body portion 46. Therefore, the mesh electrode 17 arranged within the diameter-reduced portion 45 is always in contact with water entirely, and the hydrogen generation amount can be always secured even in the case that the user sucks transversely.

The upper end edge of the mesh electrode 17 is formed by being diagonally cut along the shape of the diameter-reduced portion 45 and the opening 45c in such a manner that the whole electrode is immersed into water within the diameter-reduced portion 45 Returning back to FIG. 1, the lower end of the electrolysis tank 10 is closed by an electrolysis tank bottom 11, the electrolysis tank bottom 11 is provided with a pair or through holes into which each of the mesh electrodes 17 is inserted, and the mesh electrode 17 passes through the through hole of the electrolysis tank bottom 11 and is positioned within the diameter-reduced portion 45 by inserting the diameter-reduced portion 45 of the electrolysis tank 10 into the electrolysis tank reception portion 44 of the cover main body 1.

A description will be given of the umbrella valve 23 which is installed to the through opening 12a of the electrolysis tank lid 12 in the upper end of the electrolysis tank 10. When the screw cap 14 having an opening in its above and passing through up and down is installed to the through opening 12a, a vent filter 18 is interposed between a hole in a bottom portion of the screw cap 14 and a bottom portion of the through opening 12a, and an O-ring 21 is inserted into a downward periphery of the screw cap 14. The bent filter 18 has a function of waterproofing and dustproofing while regulating the internal pressure in the opening of the screw cap 14 with minute holes. Further, the O-ring 21 shields, from water, between an outer peripheral wall of the opening in the screw cap 14 and an inner peripheral wall of the through opening 12a.

Further, the umbrella valve 23 (made of a raw material having a flexibility such as silicone) operating in an up-and-down direction is attached to the opening of the screw cap 14, and the umbrella valve 23 is operated to move upward and fluidly connects to the inside of the electrolysis tank 10 via the through hole in the bottom portion of the screw cap 14 and the through opening 12a of the electrolysis tank lid 12 when the user sucks the nozzle 5 (mentioned later) and the negative pressure acts upward. Therefore, when the user sucks the nozzle 5, the hydrogen gas rising and accumulated within the electrolysis tank 10 is discharged to the outside. On the contrary, in a state in which the user interrupts the sucking and the negative pressure does not act, the umbrella valve 23 is operated to move down and the through hole in the bottom portion of the screw cap 14 is closed to close the discharging of the hydrogen gas within the electrolysis tank 10.

The mixer 2 is attached from the above to the electrolysis tank lid 12 to which the screw cap 14 and the umbrella valve 23 are installed. The mixer 2 has a tubular member 2a which extends downward as shown in FIG. 3, and the tubular member 2a forms a flow channel which guides upward the hydrogen gas from the umbrella valve 23, by inserting a lower end of the tubular member 2a into the opening of the screw cap 14. An O-ring 20 is provided around an outer peripheral wall of the tubular member 2a, and seals a clearance gap between the inner wall of the opening of the screw cap 14 and the tubular member 2a of the umbrella valve 20.

The mixer 2 and the electrolysis tank lid 12 are fixed by attaching lock buttons 3 and 4. Each of the lock buttons 3 and 4 is pinched in a back and forth direction (a vertical direction on the page space of FIG. 3) at the clearance gap position in the up-and-down direction between the mixer 2 and the electrolysis tank lid 12 and snaps. Further, as shown in FIG. 3, the mixer 2 is provided in its upper portion with a flow channel 2b toward the nozzle 5 direction. The flow channel 2b is connected to the flow channel formed by the tubular member 2a, and guides the hydrogen gas as shown by an arrow in FIG. 3.

A description will be given of the aromatic heater portion 32 which generates the aromatic air.

First of all, a contact terminal 37 of the battery 36 is inserted into an upper end opening of the battery reception portion 43 in the main body cover 1. The contact terminal 37 is formed by connecting a bottom portion of a large-diameter cylinder and an upper portion of a small-diameter cylinder, and the bottom portion is inserted into an opening in an upper end of the battery reception portion 43 and the electric power is supplied from the battery 36 to the aromatic heater portion 32. The contact terminal 37 is fastened to a joint 8 from the above with a cross recessed head screw 38. The joint 8 is formed in such a manner that the bottom portion of the small-diameter cylinder is connected to an approximately large-diameter discoid upper portion, and the upper portion of the contact terminal 37 and the bottom portion of the joint 8 are fitted in a nested manner.

The aromatic heater member 32 is mounted on the upper surface of the joint 8, and is pinched by the joint 8 and the mixer 2 and is fixed to the main body cover 1 when attaching the mixer 2 mentioned above. The aromatic heater member 32 is a general-purpose device, and is structured such that the air with aroma is generated in an inner portion thereof and is discharged upward when the electric power is supplied. Further, the mixer 2 is provided with a tubular member 2c which extends downward in parallel to the tubular member 2a mentioned above, and an upper end of the aromatic heater portion 32 is connected to the tubular member 2c. Therefore, the air with aroma discharged from the aromatic heater portion 32 passes through the tubular member 2c as shown by an arrow in FIG. 3, and joins together with the hydrogen gas flowing out of the flow channel 2b via the tubular member 2a so as to be flowed into the nozzle 5 and be discharged into a mouth of the user.

The nozzle 5 is structured such that the approximately large-diameter discoid member in the bottom portion is integrally connected to the tubular member in the upper portion, and the bottom portion is installed onto the opening in a top surface which is fluidly connected to the tubular member 2c of the heater portion 32 in the mixer 2. As a result, the hydrogen gas from the flow channel 2b and/or the air with aroma from the tubular member 2c is discharged to the outer portion of the upper end from the nozzle 5. An O-ring 22 is arranged in a connection portion between the bottom portion of the nozzle 5 and the mixer 2, and the connection portion is sealed.

Further, the control board 33 controls the electric power supply from the battery 36 to the aromatic heater portion 32. As mentioned above, the electric power to the mesh substrate 17 is supplied for a predetermined time by pushing the button 35 attached to the main body cover 1 three times. In the meanwhile, when the button is held down, the control board 33 connects the contact terminal 37 under a condition that the electric power supply signal is not transmitted to the mesh electrode 17, so that the electric power from the battery 36 is supplied to the aromatic heater portion 32 for a predetermined time.

Therefore, in the case that the button 35 is pushed three times and the user sucks the nozzle 5, the hydrogen gas is discharged from the nozzle 5, and the user can enjoy sucking the hydrogen gas for a predetermined time (during the light emission of the LED substrate 30). In the case that the button 35 is held down during the discharging of the hydrogen gas, the user can enjoy sucking the hydrogen gas with aroma.

The description is given above of the embodiment by exemplifying the hydrogen gas suction tool according to the present invention. However, those skilled in the art can understand that the present invention is not limited to the embodiment, but the other modified examples and improved examples can be obtained within the range which does not deviate from the scope and teaching described in the claims and the description.

### INDUSTRIAL APPLICABILITY

According to the electrolysis-type hydrogen gas suction tool of the present invention, a sufficient amount of hydrogen gas generation can be secured even in the case that the tool is inclined in a state in which moisture within the electrolysis tank is reduced, while securing a space for embedding a battery and securing the water shielding between the electrolysis tank and the battery even if the tool has the rechargeable battery and is compact and inexpensive so that the user can bring and freely carry with.

### REFERENCE SIGNS LIST

100: electrolysis-type hydrogen gas suction tool
1: main body cover (main body cover member)
2: mixer (mixing portion)
5: nozzle
10: electrolysis tank
12: electrolysis tank lid
17: mesh electrode (negative electrode)
23: umbrella valve (regulation valve)
32: aromatic heater portion (aromatic heater member)
33: control board
36: battery
42: control board
45: diameter-reduced portion
45c: partition plate
46: water storage main body portion

1. An electrolysis-type hydrogen gas suction tool comprising:
a main body cover member having a battery, a control board which controls an electric power supply from the battery, and a pair of positive and negative electrodes which are applied current in relation to a positive electrode and a negative electrode of the battery or are shielded the current application thereof on the basis of the control board;
a water storable electrolysis tank which is detachably attached to the main body cover member and has a lower portion in which the pair of positive and negative electrodes is inserted inside under the attached state;
a nozzle which has a through hole; and
a mixing portion which has a flow channel fluidly connecting the nozzle portion and an upper end of the electrolysis tank,
wherein the electrolysis tank is constructed by an upper portion and a lower portion which are fluidly connected in their inner portions and are integrally formed, has a narrowing width from a side portion at one side to a side portion at the other side in the upper portion toward a boundary between the upper portion and the lower portion, and is provided with a partition plate disposed from the vicinity of the boundary in the other side toward the one side.

2. The electrolysis-type hydrogen gas suction tool according to claim 1, wherein the pair of positive and negative electrodes is arranged over all the areas in the vertical and width directions of the lower portion of the electrolysis tank.

3. The electrolysis-type hydrogen gas suction tool according to claim 1, wherein the side portions at one side of the upper portion and the lower portion of the electrolysis tank are formed into an approximately step shape, the side portions at the other side of the upper portion and the lower portion of the electrolysis tank are continuously formed into a single side portion, and the lower portion of the electrolysis tank is fitted and inserted into an electrolysis tank reception portion in the vertical direction which is provided in a side portion of the main body cover member.

4. The electrolysis-type hydrogen gas suction tool according to claim 1, wherein the electrolysis tank is specifically formed, in a cross sectional view in the vertical direction, into the following shape:
the upper portion thereof is formed into an approximately trapezoidal shape having legs which are inclined from an upper bottom to a lower bottom in the lower portion side of the electrolysis tank by setting both sides of the electrolysis tank in the width direction respectively to the upper bottom and the lower bottom which is longer than the upper bottom;
the lower portion is formed into an approximately trapezoidal shape having legs which are inclined from an upper bottom to a lower bottom in the upper portion side of the electrolysis tank by setting both sides of the electrolysis tank in the width direction respectively to the lower bottom and the upper bottom which is longer than the lower bottom; and
the legs inclined from the upper bottom to the lower bottom in the upper portion and the lower portion of the electrolysis tank are approximately in common with the lower bottom of the upper portion and the upper bottom of the lower portion,
wherein a portion connecting the lower bottom in the lower portion from the upper bottom in the upper portion of the electrolysis tank in the leg is set to a bottom surface in the upper portion of the electrolysis tank for closing from an outside, a portion connecting the lower bottom in the upper portion from the upper bottom in the lower portion of the electrolysis tank in the leg is set to a bottom surface in the upper portion of the electrolysis tank for closing from the outside, and a partition plate is provided so as to extend to an intermediate position of the lower bottom in the lower portion of the electrolysis tank along the leg from the lower bottom in the lower portion of the electrolysis tank.

5. The electrolysis-type hydrogen gas suction tool according to claim 1, wherein a battery reception portion, the electrolysis tank reception portion and the control board of the main body cover member are arranged side by side in the vertical direction.

6. The electrolysis-type hydrogen gas suction tool according to claim 1, further comprising an aromatic heater member which generates air with aroma by an electric power supply above the battery reception portion of the main body cover member.

7. The electrolysis-type hydrogen gas suction tool according to claim 1, wherein when the control board receives an operation signal requesting the hydrogen gas generation from the user and an operation signal requesting the air with aroma generation from the user, the control board controls the electric power supply and shutting off to the pair of positive and negative electrodes and to the aromatic heater member on the basis of the respective signals.

8. The electrolysis-type hydrogen gas suction tool according to claim 7, wherein the control board controls so as to supply the electric power from the battery to the aromatic heater member under a condition that the electric power from the battery is supplied to the pair of positive and negative electrodes.

9. The electrolysis-type hydrogen gas suction tool according to claim 1, further comprising the mixing portion which is structured such as to be installed to the bottom portion of the nozzle as well as being installed to the above of the electrolysis tank, and have a hydrogen gas flow channel which fluidly connects from the inner portion of the electrolysis tank to the through hole of the nozzle.

10. The electrolysis-type hydrogen gas suction tool according to claim 9, wherein the mixing portion is installed above the electrolysis tank and above the aromatic heater member and provided with an aromatic air flow channel which fluidly connects from the aromatic heater member to the through hole of the nozzle,
wherein the hydrogen gas flow channel and the aromatic air flow channel are structured such as to be joined together and be guided to the through hole of the nozzle.

11. The electrolysis-type hydrogen gas suction tool according to claim 9, further comprising a regulation valve which is opened when negative pressure is applied to the mixing portion side from the electrolysis tank to the hydrogen gas flow channel of the mixing portion, and is closed when negative pressure is not applied.
